(19) United States
(12) Patent Application Publication
(10) Pub. No.: US 2022/0342213 A1
(43) Pub. Date: Oct. 27, 2022

(54) MISCELLANEOUS AUDIO SYSTEM APPLICATIONS

(71) Applicant: Meta Platforms Technologies, LLC, Menlo Park, CA (US)

(72) Inventors: Hao Lu, Redmond, WA (US); William Owen Brimijoin, II, Kirkland, WA (US); Christi Miller, Seattle, WA (US); Limin Zhou, Redmond, WA (US); Andrew Lovitt, Redmond, WA (US)

(21) Appl. No.: 17/860,984

(22) Filed: Jul. 8, 2022

Related U.S. Application Data
(60) Provisional application No. 63/228,751, filed on Aug. 3, 2021.
(60) Provisional application No. 63/318,917, filed on Mar. 11, 2022.
(60) Provisional application No. 63/330,873, filed on Apr. 14, 2022.
(60) Provisional application No. 63/332,593, filed on Apr. 19, 2022.

(51) Int. Cl. G02B 27/00; H04R 1/32; H04R 3/00; G06F 3/01; G02B 27/01

(52) U.S. Cl. CPC G02B 27/0093; H04R 1/326; H04R 3/005; G06F 3/012; G06F 3/013; G02B 27/0172; H04R 2430/23; G02B 2027/0138

(57) ABSTRACT

Embodiments relate to an audio system for various audio applications. The audio system registers the locations of one or more sound sources and selects the target sound source based on a hidden Markov model. A health monitoring system that integrates an audio system may use information collected by sensors to monitor an amount of social interaction of a user and predict a risk of dementia and/or hearing loss based on a model. The audio system uses a current/voltage sensor to detect electrical drive signals for determining a level of audio leakage of the audio system. Additionally, the audio system may update a video stream with an audio background based on an artificial visual background in the video stream so that the updated video stream sounds as if it originated from the user being located in a physical representation related to the background.

### CROSS-REFERENCE TO RELATED APPLICATIONS

This application claims a priority and benefit to U.S. Provisional Patent Application Ser. No. 63/228,751, filed Aug. 3, 2021, U.S. Provisional Patent Application Ser. No. 63/318,917, filed Mar. 11, 2022, U.S. Provisional Patent Application Ser. No. 63/330,873, filed Apr. 14, 2022, and U.S. Provisional Patent Application Ser. No. 63/332,593, filed Apr. 19, 2022, each of which is hereby incorporated by reference in its entirety.

### FIELD OF THE INVENTION

This disclosure relates generally to audio systems, and more specifically to relates to processing of audio content for audio systems.

### BACKGROUND

To optimally improve the signal-to-noise ratio in noisy environments requires an accurate sound source selection. Conventional sound source selection uses beamforming to identify the sound source. However, this selection method is based on the assumptions that the sound sources are spatially separated, and the beamforming can correctly identify the sound source to which the user is listening, i.e., the user's auditory attention. However, due to the inconsistency across talker layouts, the location of auditory attention cannot be accurately estimated with a simple linear model based on head movements. Therefore, a model in predicting auditory attention target in a natural conversation with only head movement of the listener is needed.

Studies show a strong association between social isolation, hearing loss, and dementia (i.e., greater social isolation and greater hearing loss is associated with greater likelihood of dementia). The causal relationship among these three constructs is unknown at this time; but researchers are actively looking for early modifiable risk factors.

Audio leakage in headphones, earbuds and hearables can impact user audio experience as well as render system calibration. Conventional audio leakage detection systems usually require microphones to capture sound and analyze the acoustic audio leakage. The additional microphones can increase the complexity of structure and routing of the audio system. For example, a conventional audio system may have an internal microphone for detecting audio leakage. This additional microphone may complicate the internal routing and may couple with mechanical vibration from the render system, thus adding up the cost of the audio system.

Backgrounds for video calls tend to be static images that only affect how a user appears to others on the call, and do not affect how the user sounds to others on the call. Conventional video conferences may provide artificial visual background, and the user may look like being in the physical location that is related to the artificial background. However, the artificial background does not have any acoustic effect, as such, the user does not sound like being in the physical location. For example, the user may look like being located in a concert hall, but still sound like being at the home office. Therefore, the conventional artificial background does not provide a full immersed user experience.

### SUMMARY

Embodiments of the present disclosure relate to a method for determining a target sound source. The method comprises: registering locations of one or more sound sources relative to a user's location; detecting, by one or more sensors, a head movement of the user; determining a target sound source from the one or more sound sources using a hidden Markov model (HMM) based on the detected head movement and the locations of the one or more sound sources; and selecting auditory signals from the target sound source as an input to the user.

Embodiments of the present disclosure further relate to a method for predicting risk of dementia by tracking user social activities. The method comprises: capturing, by the one or more sensors, information describing a social interaction of a user over a given period of time; determining an amount of the social interaction of the user for the given period of time based in part on the captured information; predicting a risk of dementia of the user using the amount of social interaction and a model; generating a recommendation for future social interaction of the user based in part on the predicted risk; and presenting the recommendation to the user.

Embodiments of the present disclosure further relate to a method for detecting audio leakage of an audio system. The method comprises: detecting, via an UV sensor of an audio system, an electrical drive signal provided to a speaker of the audio system having a fixed acoustic volume; determining, via a controller of the audio system, a level of audio leakage based on the detected electrical drive signal and a model; and responsive to the level of audio leakage being above a threshold value, alerting, via the audio system, a user to the audio leakage.

Embodiments of the present disclosure further relate to a method of augmenting audio background based on artificial visual background. The method comprises: receiving an audio stream from a sound source and a background image that is associated with one or more acoustic parameters. The acoustic parameters describe an acoustic effect a physical representation related to the background image has on audio. The method further comprises updating the audio stream based on the one or more acoustic parameters to generate an updated audio stream; and providing the updated audio stream to a communication device. The communication device presents the updated audio stream having the acoustic effect as if the sound source is located in the physical representation related to the background image.

### BRIEF DESCRIPTION OF THE DRAWINGS

FIG. 1A is a perspective view of a headset implemented as an eyewear device, in accordance with one or more embodiments.

FIG. 1B is a perspective view of a headset implemented as a head-mounted display, in accordance with one or more embodiments.

FIG. 2 is a block diagram of an audio system, in accordance with one or more embodiments.

FIG. 3A is an exemplary implementation scenario of the sound source selection method based on HMM in a natural conversation group, in accordance with one or more embodiments.

FIG. 3B shows an exemplary relationship between true talker directions and HMM emission means, in accordance with one or more embodiments.

FIG. 4 is a flowchart of a method for predicting risk of dementia by tracking user social activity, in accordance with one or more embodiments.

FIG. 5 illustrates an example audio system with an I/V sensor to detect audio leakage, in accordance with one or more embodiments.

FIG. 6 is a flowchart of a method of augmenting audio background based on artificial visual background, in accordance with one or more embodiments.

FIG. 7 is a system that includes a headset, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

Embodiments of the present disclosure relate to an audio system for various applications. In some embodiments, the audio system determines a target sound source based on a hidden Markov model (HMM). The audio system first registers the locations of one or more sound sources and then selects the target sound source based on HMM. This sound source selection method can significantly reduce error in identifying the target talker in a group conversation and can be generalized to group conversation with more talkers. In some embodiments, the audio system uses information collected by the sensors to monitor an amount of social interaction of a user. As there is a strong association between social isolation, hearing loss, and dementia. The system can analyze user's social interaction to predict a risk of dementia and/or hearing loss based on a model. And then the system can generate a recommendation for future social interaction of the user based in part on the predicted risk. In some embodiments, the audio system may use a current/voltage sensor to detect audio leakage of the audio system. Based on the detected electrical drive signals, the audio system can determine a level of audio leakage and alert the user to the audio leakage. In some other embodiments, the audio system may augment audio background based on artificial visual background in a video stream. The background may have associated acoustic parameters whose values describe effects a physical representation of the background image has on audio. The audio system determines the acoustic parameters related to the background, and updates the stream in accordance with the acoustic parameters so that the updated audio stream sounds as if it originated from the user being located in the physical representation related to the background.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to create content in an artificial reality and/or are otherwise used in an artificial reality. The artificial reality system that provides the artificial reality content may be implemented on various platforms, including a wearable device (e.g., headset) connected to a host computer system, a standalone wearable device (e.g., headset), a mobile device or computing system, or any other hardware platform capable of providing artificial reality content to one or more viewers.

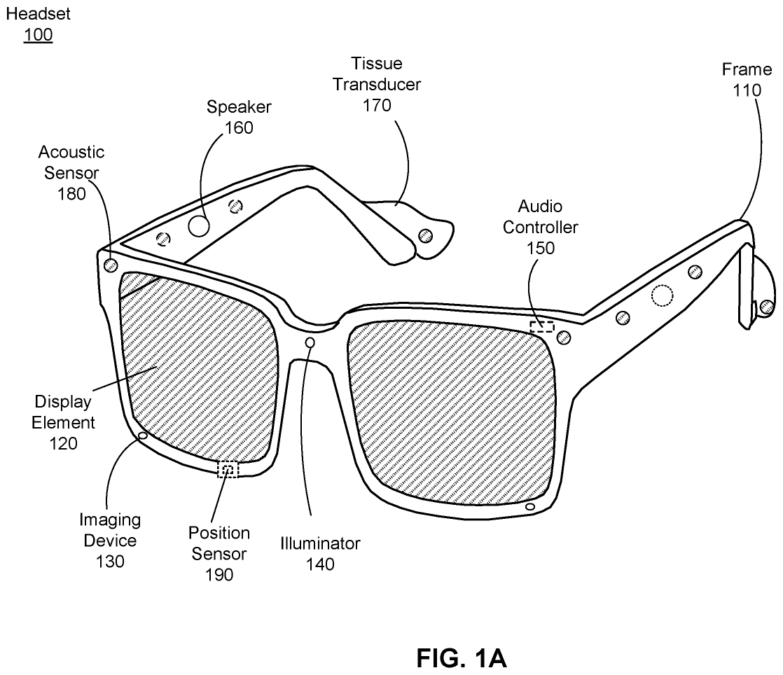

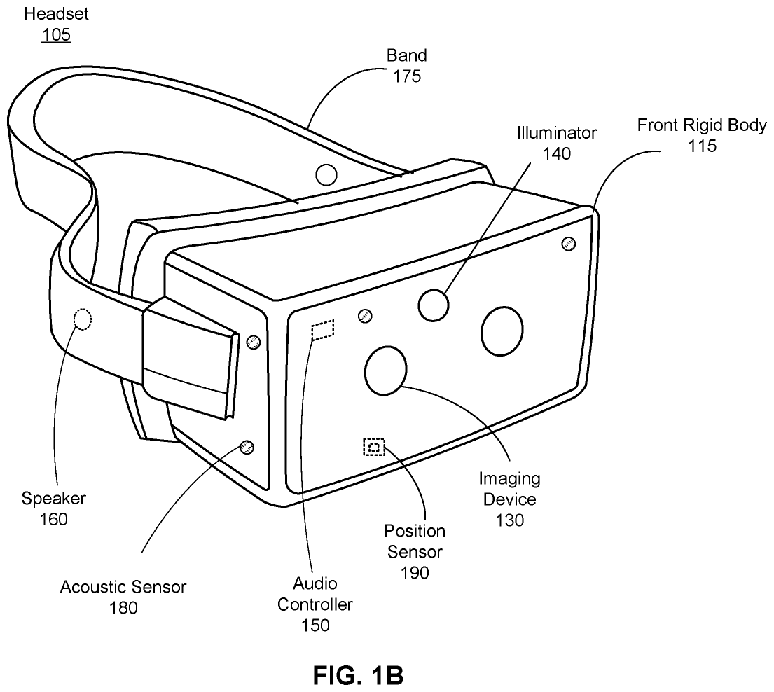

FIG. 1A is a perspective view of a headset 100 implemented as an eyewear device, in accordance with one or more embodiments. In some embodiments, the eyewear device is a near eye display (NED). In general, the headset 100 may be worn on the face of a user such that content (e.g., media content) is presented using a display assembly and/or an audio system. However, the headset 100 may also be used such that media content is presented to a user in a different manner. Examples of media content presented by the headset 100 include one or more images, video, audio, or some combination thereof. The headset 100 includes a frame, and may include, among other components, a display assembly including one or more display elements 120, a depth camera assembly (DCA), an audio system, and a position sensor 190. While FIG. 1A illustrates the components of the headset 100 in example locations on the headset 100, the components may be located elsewhere on the headset 100, on a peripheral device paired with the headset 100, or some combination thereof. Similarly, there may be more or fewer components on the headset 100 than what is shown in FIG. 1A.

The frame 110 holds the other components of the headset 100. The frame 110 includes a front part that holds the one or more display elements 120 and end pieces (e.g., temples) to attach to a head of the user. The front part of the frame 110 bridges the top of a nose of the user. The length of the end pieces may be adjustable (e.g., adjustable temple length) to fit different users. The end pieces may also include a portion that curls behind the ear of the user (e.g., temple tip, ear piece).

The one or more display elements 120 provide light to a user wearing the headset 100. As illustrated the headset includes a display element 120 for each eye of a user. In some embodiments, a display element 120 generates image light that is provided to an eyebox of the headset 100. The eyebox is a location in space that an eye of user occupies while wearing the headset 100. For example, a display element 120 may be a waveguide display. A waveguide display includes a light source (e.g., a two-dimensional source, one or more line sources, one or more point sources, etc.) and one or more waveguides. Light from the light source is in-coupled into the one or more waveguides which outputs the light in a manner such that there is pupil replication in an eyebox of the headset 100. In-coupling and/or outcoupling of light from the one or more waveguides may be done using one or more diffraction gratings. In some embodiments, the waveguide display includes a scanning element (e.g., waveguide, mirror, etc.) that scans light from the light source as it is in-coupled into the one or more waveguides. Note that in some embodiments, one or both of the display elements 120 are opaque and do not transmit light from a local area around the headset 100. The local area is the area surrounding the headset 100. For example, the local area may be a room that a user wearing the headset 100 is inside, or the user wearing the headset 100 may be outside and the local area is an outside area. In this context, the headset 100 generates VR content. Alternatively, in some embodiments, one or both of the display elements 120 are at least partially transparent, such that light from the local area may be combined with light from the one or more display elements to produce AR and/or MR content.

In some embodiments, a display element 120 does not generate image light, and instead is a lens that transmits light from the local area to the eyebox. For example, one or both of the display elements 120 may be a lens without correction (non-prescription) or a prescription lens (e.g., single vision, bifocal and trifocal, or progressive) to help correct for defects in a user's eyesight. In some embodiments, the display element 120 may be polarized and/or tinted to protect the user's eyes from the sun.

In some embodiments, the display element 120 may include an additional optics block (not shown). The optics block may include one or more optical elements (e.g., lens, Fresnel lens, etc.) that direct light from the display element 120 to the eyebox. The optics block may, e.g., correct for aberrations in some or all of the image content, magnify some or all of the image, or some combination thereof.

The DCA determines depth information for a portion of a local area surrounding the headset 100. The DCA includes one or more imaging devices 130 and a DCA controller (not shown in FIG. 1A), and may also include an illuminator 140. In some embodiments, the illuminator 140 illuminates a portion of the local area with light. The light may be, e.g., structured light (e.g., dot pattern, bars, etc.) in the infrared (IR), IR flash for time-of-flight, etc. In some embodiments, the one or more imaging devices 130 capture images of the portion of the local area that include the light from the illuminator 140. As illustrated, FIG. 1A shows a single illuminator 140 and two imaging devices 130. In alternate embodiments, there is no illuminator 140 and at least two imaging devices 130.

The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 140), some other technique to determine depth of a scene, or some combination thereof.

The DCA may include an eye tracking unit that determines eye tracking information. The eye tracking information may comprise information about a position and an orientation of one or both eyes (within their respective eye-boxes). The eye tracking unit may include one or more cameras. The eye tracking unit estimates an angular orientation of one or both eyes based on images captures of one or both eyes by the one or more cameras. In some embodiments, the eye tracking unit may also include one or more illuminators that illuminate one or both eyes with an illumination pattern (e.g., structured light, glints, etc.). The eye tracking unit may use the illumination pattern in the captured images to determine the eye tracking information. The headset 100 may prompt the user to opt in to allow operation of the eye tracking unit. For example, by opting in the headset 100 may detect, store, images of the user's any or eye tracking information of the user.

The audio system provides audio content. The audio system includes a transducer array, a sensor array, and an audio controller 150. However, in other embodiments, the audio system may include different and/or additional components. Similarly, in some cases, functionality described with reference to the components of the audio system can be distributed among the components in a different manner than is described here. For example, some or all of the functions of the controller may be performed by a remote server.

The transducer array presents sound to user. The transducer array includes a plurality of transducers. A transducer may be a speaker 160 or a tissue transducer 170 (e.g., a bone conduction transducer or a cartilage conduction transducer). Although the speakers 160 are shown exterior to the frame 110, the speakers 160 may be enclosed in the frame 110. In some embodiments, instead of individual speakers for each ear, the headset 100 includes a speaker array comprising multiple speakers integrated into the frame 110 to improve directionality of presented audio content. The tissue transducer 170 couples to the head of the user and directly vibrates tissue (e.g., bone or cartilage) of the user to generate sound. The number and/or locations of transducers may be different from what is shown in FIG. 1A.

The sensor array detects sounds within the local area of the headset 100. The sensor array includes a plurality of acoustic sensors 180. An acoustic sensor 180 captures sounds emitted from one or more sound sources in the local area (e.g., a room). Each acoustic sensor is configured to detect sound and convert the detected sound into an electronic format (analog or digital). The acoustic sensors 180 may be acoustic wave sensors, microphones, sound transducers, or similar sensors that are suitable for detecting sounds.

In some embodiments, one or more acoustic sensors 180 may be placed in an ear canal of each ear (e.g., acting as binaural microphones). In some embodiments, the acoustic sensors 180 may be placed on an exterior surface of the headset 100, placed on an interior surface of the headset 100, separate from the headset 100 (e.g., part of some other device), or some combination thereof. The number and/or locations of acoustic sensors 180 may be different from what is shown in FIG. 1A. For example, the number of acoustic detection locations may be increased to increase the amount of audio information collected and the sensitivity and/or accuracy of the information. The acoustic detection locations may be oriented such that the microphone is able to detect sounds in a wide range of directions surrounding the user wearing the headset 100.

The audio controller 150 processes information from the sensor array that describes sounds detected by the sensor array. The audio controller 150 may comprise a processor and a computer-readable storage medium. The audio controller 150 may be configured to generate direction of arrival (DOA) estimates, generate acoustic transfer functions (e.g., array transfer functions and/or head-related transfer functions), track the location of sound sources, form beams in the direction of sound sources, classify sound sources, generate sound filters for the speakers 160, or some combination thereof.

The position sensor 190 generates one or more measurement signals in response to motion of the headset 100. The position sensor 190 may be located on a portion of the frame 110 of the headset 100. The position sensor 190 may include an inertial measurement unit (IMU). Examples of position sensor 190 include: one or more accelerometers, one or more gyroscopes, one or more magnetometers, another suitable type of sensor that detects motion, a type of sensor used for error correction of the IMU, or some combination thereof. The position sensor 190 may be located external to the IMU, internal to the IMU, or some combination thereof.

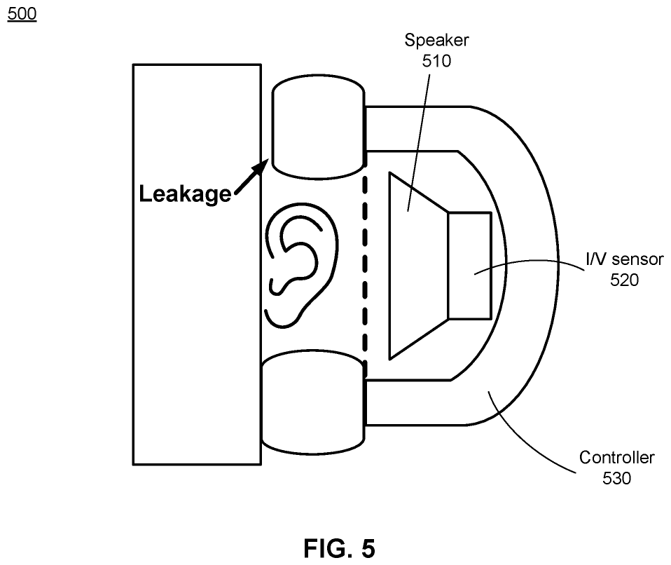

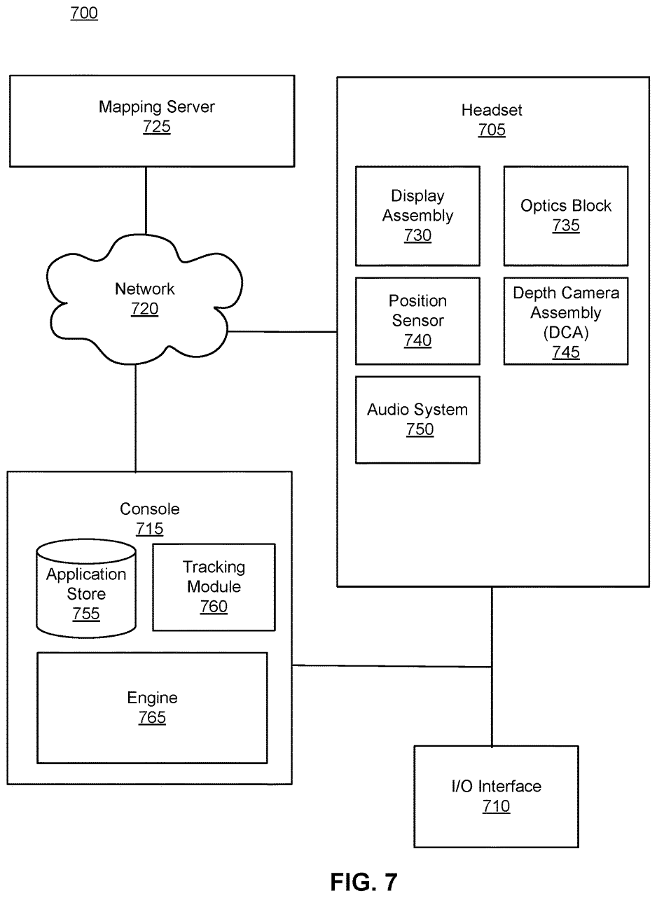

In some embodiments, the headset 100 may provide for simultaneous localization and mapping (SLAM) for a position of the headset 100 and updating of a model of the local area. For example, the headset 100 may include a passive camera assembly (PCA) that generates color image data. The PCA may include one or more RGB cameras that capture images of some or all of the local area. In some embodiments, some or all of the imaging devices 130 of the DCA may also function as the PCA. The images captured by the PCA and the depth information determined by the DCA may be used to determine parameters of the local area, generate a model of the local area, update a model of the local area, or some combination thereof. Furthermore, the position sensor 190 tracks the position (e.g., location and pose) of the headset 100 within the room. Additional details regarding the components of the headset 100 are discussed below in connection with FIG. 5 and FIG. 7.

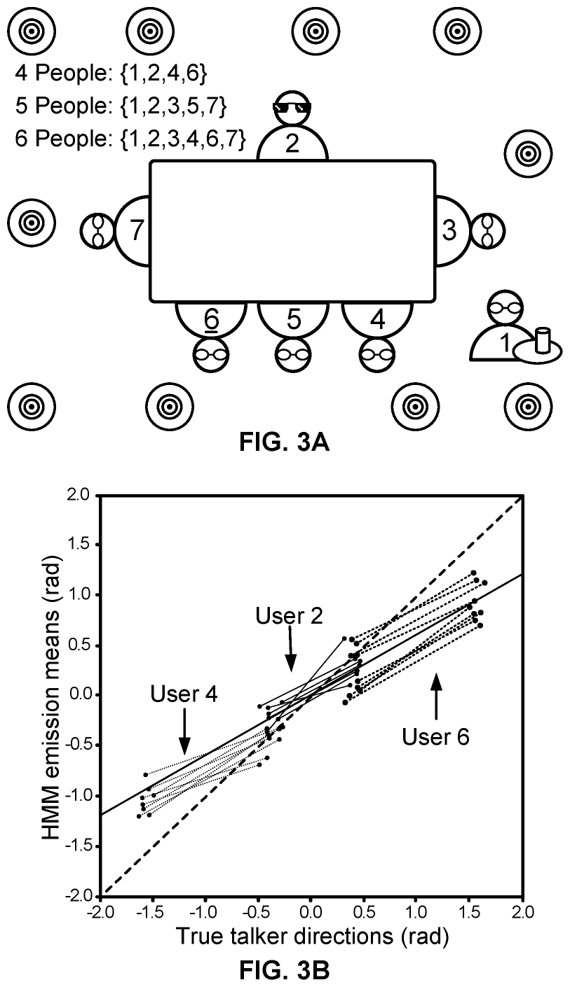

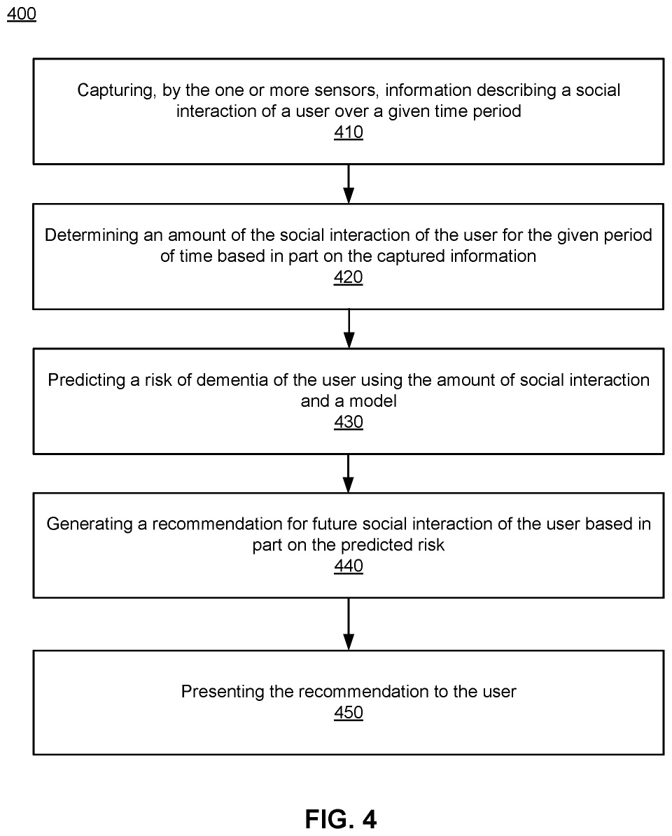

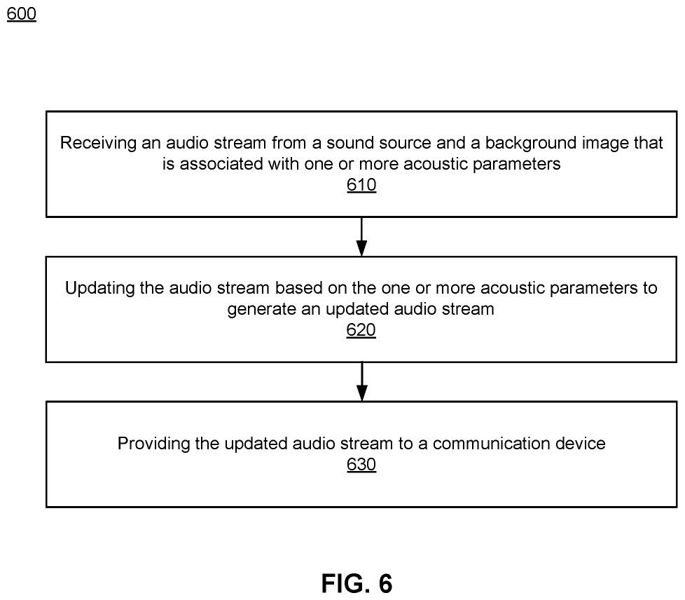

The headset 100 may be configured to register sound sources and/or detect user behaviors by using the display element 120, imaging device 130, acoustic sensor 180, position sensor 190, and/or other components. Based on the detected information, the headset 100 may determine a target sound source and select auditory signals from the target sound source as an input to the user (e.g., as described below with regard to FIG. 3A and FIG. 3B). The headset 100 may also be integrated as part of a health monitoring system (e.g., as described below with regard to FIG. 4). The health monitoring system captures information describing a social interaction of a user, determines an amount of the user's social interaction, and predicts a risk of dementia and/hearing loss of the user. Additionally, the headset 100 may comprise a current/voltage sensor to detect audio leakage as shown in FIG. 5. Further, the headset 100 may be configured to augment audio background based on an artificial visual background in a video stream (e.g., as described below with regard to FIG. 6).

FIG. 1B is a perspective view of a headset 105 implemented as a HMD, in accordance with one or more embodiments. In embodiments that describe an AR system and/or a MR system, portions of a front side of the HMD are at least partially transparent in the visible band (˜380 nm to 750 nm), and portions of the HMD that are between the front side of the HMD and an eye of the user are at least partially transparent (e.g., a partially transparent electronic display). The HMD includes a front rigid body 115 and a band 175. The headset 105 includes many of the same components described above with reference to FIG. 1A, but modified to integrate with the HMD form factor. For example, the HMD includes a display assembly, a DCA, an audio system, and a position sensor 190. FIG. 1B shows the illuminator 140, a plurality of the speakers 160, a plurality of the imaging devices 130, a plurality of acoustic sensors 180, and the position sensor 190. The speakers 160 may be located in various locations, such as coupled to the band 175 (as shown), coupled to front rigid body 115, or may be configured to be inserted within the ear canal of a user.

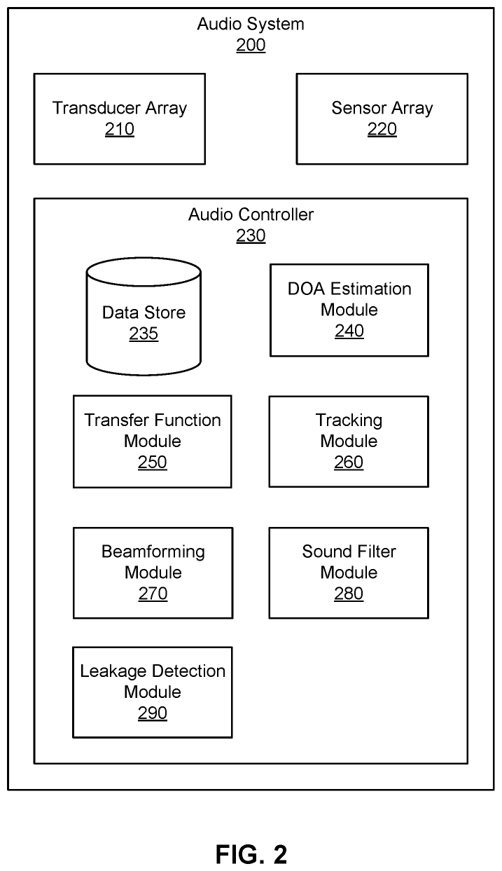

FIG. 2 is a block diagram of an audio system 200, in accordance with one or more embodiments. The audio system in FIG. 1A or FIG. 1B may be an embodiment of the audio system 200. The audio system 200 generates one or more acoustic transfer functions for a user. The audio system 200 may then use the one or more acoustic transfer functions to generate audio content for the user. In the embodiment of FIG. 2, the audio system 200 includes a transducer array 210, a sensor array 220, and an audio controller 230. Some embodiments of the audio system 200 have different components than those described here. Similarly, in some cases, functions can be distributed among the components in a different manner than is described here.

The transducer array 210 is configured to present audio content. The transducer array 210 includes a plurality of transducers. A transducer is a device that provides audio content. A transducer may be, e.g., a speaker (e.g., the speaker 160), a tissue transducer (e.g., the tissue transducer 170), some other device that provides audio content, or some combination thereof. A tissue transducer may be configured to function as a bone conduction transducer or a cartilage conduction transducer. The transducer array 210 may present audio content via air conduction (e.g., via one or more speakers), via bone conduction (via one or more bone conduction transducer), via cartilage conduction audio system (via one or more cartilage conduction transducers), or some combination thereof. In some embodiments, the transducer array 210 may include one or more transducers to cover different parts of a frequency range. For example, a piezoelectric transducer may be used to cover a first part of a frequency range and a moving coil transducer may be used to cover a second part of a frequency range.

The bone conduction transducers generate acoustic pressure waves by vibrating bone/tissue in the user's head. A bone conduction transducer may be coupled to a portion of a headset, and may be configured to be behind the auricle coupled to a portion of the user's skull. The bone conduction transducer receives vibration instructions from the audio controller 230, and vibrates a portion of the user's skull based on the received instructions. The vibrations from the bone conduction transducer generate a tissue-borne acoustic pressure wave that propagates toward the user's cochlea, bypassing the eardrum.

The cartilage conduction transducers generate acoustic pressure waves by vibrating one or more portions of the auricular cartilage of the ears of the user. A cartilage conduction transducer may be coupled to a portion of a headset, and may be configured to be coupled to one or more portions of the auricular cartilage of the ear. For example, the cartilage conduction transducer may couple to the back of an auricle of the ear of the user. The cartilage conduction transducer may be located anywhere along the auricular cartilage around the outer ear (e.g., the pinna, the tragus, some other portion of the auricular cartilage, or some combination thereof). Vibrating the one or more portions of auricular cartilage may generate: airborne acoustic pressure waves outside the ear canal; tissue born acoustic pressure waves that cause some portions of the ear canal to vibrate thereby generating an airborne acoustic pressure wave within the ear canal; or some combination thereof. The generated airborne acoustic pressure waves propagate down the ear canal toward the ear drum.

The transducer array 210 generates audio content in accordance with instructions from the audio controller 230. In some embodiments, the audio content is spatialized. Spatialized audio content is audio content that appears to originate from a particular direction and/or target region (e.g., an object in the local area and/or a virtual object). For example, spatialized audio content can make it appear that sound is originating from a virtual singer across a room from a user of the audio system 200. The transducer array 210 may be coupled to a wearable device (e.g., the headset 100 or the headset 105). In alternate embodiments, the transducer array 210 may be a plurality of speakers that are separate from the wearable device (e.g., coupled to an external console). In some embodiments, the transducer array 210 is configured to update an audio stream with acoustic parameters so that the updated audio stream sounds as if it originated from a user being located in a physical representation related to a background, and the background is associated with the acoustic parameters (as shown in FIG. 6).

The sensor array 220 detects sounds within a local area surrounding the sensor array 220. The sensor array 220 may include a plurality of acoustic sensors that each detect air pressure variations of a sound wave and convert the detected sounds into an electronic format (analog or digital). The plurality of acoustic sensors may be positioned on a headset (e.g., headset 100 and/or the headset 105), on a user (e.g., in an ear canal of the user), on a neckband, or some combination thereof. An acoustic sensor may be, e.g., a microphone, a vibration sensor, an accelerometer, or any combination thereof. In some embodiments, the sensor array 220 is configured to monitor the audio content generated by the transducer array 210 using at least some of the plurality of acoustic sensors. Increasing the number of sensors may improve the accuracy of information (e.g., directionality) describing a sound field produced by the transducer array 210 and/or sound from the local area.

The audio controller 230 controls operation of the audio system 200. In the embodiment of FIG. 2, the audio controller 230 includes a data store 235, a DOA estimation module 240, a transfer function module 250, a tracking module 260, a beamforming module 270, a sound filter module 280, and a leakage detection module 290. The audio controller 230 may be located inside a headset, in some embodiments. Some embodiments of the audio controller 230 have different components than those described here. Similarly, functions can be distributed among the components in different manners than described here. For example, some functions of the controller may be performed external to the headset. The user may opt in to allow the audio controller 230 to transmit data captured by the headset to systems external to the headset, and the user may select privacy settings controlling access to any such data.

The data store 235 stores data for use by the audio system 200. Data in the data store 235 may include sounds recorded in the local area of the audio system 200, audio content, head-related transfer functions (HRTFs), transfer functions for one or more sensors, array transfer functions (ATFs) for one or more of the acoustic sensors, sound source locations, virtual model of local area, direction of arrival estimates, sound filters, and other data relevant for use by the audio system 200, or any combination thereof. The data store 235 may be implemented as a non-transitory computer-readable storage medium.

The user may opt-in to allow the data store 235 to record data captured by the audio system 200. In some embodiments, the audio system 200 may employ always on recording, in which the audio system 200 records all sounds captured by the audio system 200 in order to improve the experience for the user. The user may opt in or opt out to allow or prevent the audio system 200 from recording, storing, or transmitting the recorded data to other entities.

The DOA estimation module 240 is configured to localize sound sources in the local area based in part on information from the sensor array 220. Localization is a process of determining where sound sources are located relative to the user of the audio system 200. The DOA estimation module 240 performs a DOA analysis to localize one or more sound sources within the local area. The DOA analysis may include analyzing the intensity, spectra, and/or arrival time of each sound at the sensor array 220 to determine the direction from which the sounds originated. In some cases, the DOA analysis may include any suitable algorithm for analyzing a surrounding acoustic environment in which the audio system 200 is located. In some embodiments, the DOA estimation module 240 may register the locations of one or more sound sources. The registered sound sources then can be selected as a target sound source based on the user behavior (as described in FIG. 3A and FIG. 3B).

For example, the DOA analysis may be designed to receive input signals from the sensor array 220 and apply digital signal processing algorithms to the input signals to estimate a direction of arrival. These algorithms may include, for example, delay and sum algorithms where the input signal is sampled, and the resulting weighted and delayed versions of the sampled signal are averaged together to determine a DOA. A least mean squared (LMS) algorithm may also be implemented to create an adaptive filter. This adaptive filter may then be used to identify differences in signal intensity, for example, or differences in time of arrival. These differences may then be used to estimate the DOA. In another embodiment, the DOA may be determined by converting the input signals into the frequency domain and selecting specific bins within the time-frequency (TF) domain to process. Each selected TF bin may be processed to determine whether that bin includes a portion of the audio spectrum with a direct path audio signal. Those bins having a portion of the direct-path signal may then be analyzed to identify the angle at which the sensor array 220 received the direct-path audio signal. The determined angle may then be used to identify the DOA for the received input signal. Other algorithms not listed above may also be used alone or in combination with the above algorithms to determine DOA.

In some embodiments, the DOA estimation module 240 may also determine the DOA with respect to an absolute position of the audio system 200 within the local area. The position of the sensor array 220 may be received from an external system (e.g., some other component of a headset, an artificial reality console, a mapping server, a position sensor (e.g., the position sensor 190), etc.). The external system may create a virtual model of the local area, in which the local area and the position of the audio system 200 are mapped. The received position information may include a location and/or an orientation of some or all of the audio system 200 (e.g., of the sensor array 220). The DOA estimation module 240 may update the estimated DOA based on the received position information.

The transfer function module 250 is configured to generate one or more acoustic transfer functions. Generally, a transfer function is a mathematical function giving a corresponding output value for each possible input value. Based on parameters of the detected sounds, the transfer function module 250 generates one or more acoustic transfer functions associated with the audio system. The acoustic transfer functions may be array transfer functions (ATFs), head-related transfer functions (HRTFs), other types of acoustic transfer functions, or some combination thereof. An ATF characterizes how the microphone receives a sound from a point in space.

An ATF includes a number of transfer functions that characterize a relationship between the sound source and the corresponding sound received by the acoustic sensors in the sensor array 220. Accordingly, for a sound source there is a corresponding transfer function for each of the acoustic sensors in the sensor array 220. And collectively the set of transfer functions is referred to as an ATF. Accordingly, for each sound source there is a corresponding ATF. Note that the sound source may be, e.g., someone or something generating sound in the local area, the user, or one or more transducers of the transducer array 210. The ATF for a particular sound source location relative to the sensor array 220 may differ from user to user due to a person's anatomy (e.g., ear shape, shoulders, etc.) that affects the sound as it travels to the person's ears. Accordingly, the ATFs of the sensor array 220 are personalized for each user of the audio system 200.

In some embodiments, the transfer function module 250 determines one or more HRTFs for a user of the audio system 200. The HRTF characterizes how an ear receives a sound from a point in space. The HRTF for a particular source location relative to a person is unique to each ear of the person (and is unique to the person) due to the person's anatomy (e.g., ear shape, shoulders, etc.) that affects the sound as it travels to the person's ears. In some embodiments, the transfer function module 250 may determine HRTFs for the user using a calibration process. In some embodiments, the transfer function module 250 may provide information about the user to a remote system. The user may adjust privacy settings to allow or prevent the transfer function module 250 from providing the information about the user to any remote systems. The remote system determines a set of HRTFs that are customized to the user using, e.g., machine learning, and provides the customized set of HRTFs to the audio system 200.

The tracking module 260 is configured to track locations of one or more sound sources. The tracking module 260 may compare current DOA estimates and compare them with a stored history of previous DOA estimates. In some embodiments, the audio system 200 may recalculate DOA estimates on a periodic schedule, such as once per second, or once per millisecond. The tracking module may compare the current DOA estimates with previous DOA estimates, and in response to a change in a DOA estimate for a sound source, the tracking module 260 may determine that the sound source moved. In some embodiments, the tracking module 260 may detect a change in location based on visual information received from the headset or some other external source. The tracking module 260 may track the movement of one or more sound sources over time. The tracking module 260 may store values for a number of sound sources and a location of each sound source at each point in time. In response to a change in a value of the number or locations of the sound sources, the tracking module 260 may determine that a sound source moved. The tracking module 260 may calculate an estimate of the localization variance. The localization variance may be used as a confidence level for each determination of a change in movement.

The beamforming module 270 is configured to process one or more ATFs to selectively emphasize sounds from sound sources within a certain area while deemphasizing sounds from other areas. In analyzing sounds detected by the sensor array 220, the beamforming module 270 may combine information from different acoustic sensors to emphasize sound associated from a particular region of the local area while deemphasizing sound that is from outside of the region. The beamforming module 270 may isolate an audio signal associated with sound from a particular sound source from other sound sources in the local area based on, e.g., different DOA estimates from the DOA estimation module 240 and the tracking module 260. The beamforming module 270 may thus selectively analyze discrete sound sources in the local area. In some embodiments, the beamforming module 270 may enhance a signal from a sound source. For example, the beamforming module 270 may apply sound filters which eliminate signals above, below, or between certain frequencies. Signal enhancement acts to enhance sounds associated with a given identified sound source relative to other sounds detected by the sensor array 220. In some embodiments, the beamforming module 270 is configured to select auditory signals from a target sound source as an input to the user (as shown in FIG. 3A and FIG. 3B).

The sound filter module 280 determines sound filters for the transducer array 210. In some embodiments, the sound filters cause the audio content to be spatialized, such that the audio content appears to originate from a target region. The sound filter module 280 may use HRTFs and/or acoustic parameters to generate the sound filters. The acoustic parameters describe acoustic properties of the local area. The acoustic parameters may include, e.g., a reverberation time, a reverberation level, a room impulse response, etc. In some embodiments, the sound filter module 280 calculates one or more of the acoustic parameters. In some embodiments, the sound filter module 280 requests the acoustic parameters from a mapping server (e.g., as described below with regard to FIG. 7).

The sound filter module 280 provides the sound filters to the transducer array 210. In some embodiments, the sound filters may cause positive or negative amplification of sounds as a function of frequency.

The leakage detection module 290 is configured to receive detected electrical signals from an I/V sensor. Based on the electrical signals, the leakage detection module 290 may determine whether there is an audio leakage in the audio system using a model. In some embodiments, the leakage detection module 290 may further analyze the causation of the audio leakage so that the audio system may provide an alert and/or recommendation to the user.

### Sound Source Selection Based on Head Movements in Natural Group Conversation

Embodiments of the present disclosure may include or be implemented in conjunction with an audio system that provides spatialized audio content. The audio system may be part of a headset. In some embodiments, the headset may be an artificial reality headset (e.g., presents content in virtual reality, augmented reality, and/or mixed reality). The audio system may use the method provided in embodiments herein to render spatialized audio content to users through the headset. Spatialized audio content is audio content that appears to originate from a particular direction and/or target region (e.g., an object in the local area and/or a virtual object).

Group conversation is an important form of daily social interaction, and it is commonly conducted in noisy environments such as restaurants and classrooms, which can affect the ease of communication. A typical approach is to improve signal-to-noise (SNR) ratio, for example, by using beamforming, which is frequently applied in modern hearing aids. It is designed to enhance the sound from one direction and attenuate noise from other directions. Using beamforming to improve the SNR ratio requires two important assumptions: 1) the sound sources are spatially separated, and 2) the beam is pointed correctly with respect to the sound source to which the user is listening. Thus, to optimally improve the SNR ratio in noisy environments one must correctly identify what is signal and what is noise. When the direction of beamformer does not align well with auditory attention, the user will not receive optimal SNR benefit and may experience difficulty when trying to orient towards the desired sound source.

To correctly identify user's attended sound source requires a source selection model that reflects user's auditory attention. Head movement is a pragmatic choice, as it can be conveniently estimated with inertial measurement units (IMU) and cameras on wearable devices. Taking a group conversation for example, the talker to whom the listener is attending needs to be identified as a sound source, and head movements of listeners during group conversation can provide one potential cue of auditory attention.

However, the head orientation may not directly reflect the true location of auditory target. The angle between head orientation and torso midline and the angle between target location and torso midline was found to approximately follow a linear relationship when orienting towards a sound source in lab setting. However, such a linear model may be unstable due to different room layouts, different talker positions, and variations caused by individual differences. Previous studies have demonstrated that head orientation systematically undershoots listening targets in a simple linear relationship between the true location of target talker and listener's head orientation. Additionally, predicting real-time target location purely based on head orientation is also very challenging.

Without prior knowledge of the auditory scene, the location of auditory attention may need to be inferred by sophisticated statistical modeling, such as a regression model as a vector in space. However, when the locations of possible auditory targets are available, the problem can be simplified to selecting a discrete target from a finite number of options. As the locations of talkers are usually bounded during group conversation, they can be registered through cameras or a microphone array on a wearable device. Once the number and locations of possible target talkers are identified, the source selection can be simplified as a classification problem, where discrete auditory attention states can be decoded from continuous head movements.

A sound source selection method based on a hidden Markov model (HMM) is presented herein. This method includes source registration and source selection. The possible target locations are first registered through information from environment sensors, e.g., cameras and microphone array, and the current attended target is selected through measured user's behavior, e.g., head movement. Real-time head movements are converted as a prediction of the target of a listener's auditory attention based on the HMM. This sound source selection method can significantly reduce error in identifying the target talker in a group conversation and can be generalized to group conversation with more talkers.

FIG. 3A is an exemplary implementation scenario of the sound source selection method based on HMM in a natural conversation group, in accordance with one or more embodiments. The process shown in FIG. 3A may be performed by components of an audio system (e.g., audio system 200). Other entities may perform some or all of the steps in FIG. 3A in other embodiments. Embodiments may include different and/or additional steps, or perform the steps in different orders.

As shown in FIG. 3A, users 1-7 participate in a natural group conversation. User 1 may be a standing host and users 2-7 may be sitting around a table. The users may wear audio systems 200 with an egocentric camera and a microphone array, shown as a color-filled glasses on user 2 as an example. The conversation may include any kind of natural group conversation, for example, introductions, ordering food, solving puzzles, playing games, reading sentences, etc. In some embodiments, all users may participate the conversation and talk to each other during the conversation; and in some other embodiments, only part of the user may participate in the conversation. In one example, 4 users (users 1, 2, 4, and 6) participate in the conversation; in another example, 5 users (users 1, 2, 3, 5, and 7) may participate in the conversation; and in yet another example, 6 users (users 1, 2, 3, 4, 6, and 7) may participate in the conversation. The circular rings in the background of FIG. 3A may indicate noise sound sources. In some embodiments, the background noise may be at some fixed level, for example, 71 dB SPL.

To determine a target sound source based on listener's head movement during group conversation, different source selection models can be used. One model is based simply on the linear relationship between target location and head orientation of the listener. Another model may be based on an HMM with known target locations. The performance of the linear relationship model and the HMM model can be compared with respect to the true location of the target sound source (e.g., talkers in the conversation).

The conversation may be recorded. The head location and orientation of all sitting users (for example, users 2, 3, 4, 5, 6, 7 in FIG. 3A) may be recorded at 20-Hz sample frequency. The head movements of the users can be extracted from the video, the manually-labelled speech actives, the egocentric video, and the audio recording. The true location of the talkers may be identified based on manual annotation on their real-time head location captured in the video recording. The true auditory attention target can be identified by manually labeled speech activity segments and further annotated based on the audio and video recording.

In one example, 4 users (users 1, 2, 4, and 6) participate in the conversation with two of the users considered as target talkers. The quaternions can be converted to Euler angles following the rotation order YXZ (e.g., from the view of user 2 in FIG. 3A, positive X points left; positive Y points upwards; and positive Z points forward) to obtain the yaw, pitch, and roll movements of head during the conversation. As all target sound sources are approximately on the same horizontal plane, only the yaw movements of head may be used in the analysis. Zero yaw angle can be defined as pointing towards positive or negative Z axis depending on the location of user.

To analyze the relationship between head movement distribution and the true location of target talkers, the yaw head movement of all users is individually fitted using HMIs. For each user, the HMIs may utilize two hidden states corresponding to focusing on one of the two target talkers, so the Gaussian emission functions of two hidden states correspond to the distribution of head orientation when focusing on one of the two target talkers, and the means of emission functions can be used to represent the overall head orientation. The performance of the fitted HMM and head orientation can be quantified as the yaw angle error between the predicted target direction and the true location of the attended target talker. The average locations of target talkers through the entire conversation can be calculated and used to convert the hidden states of HMM to a yaw angle estimate.

FIG. 3B shows an exemplary relationship between true talker directions and HMM emission means, in accordance with one or more embodiments. Data representing users 2, 4, and 6 are shown in the graph. The dashed line represents perfect linear relationship between the true location of the target talker and the user's head orientation without undershooting, and the solid line represents the linear relationship with undershooting revealed in the linear relationship model. The means of HMM emission functions can be computed for each user in the 4-people session. The slope and intercept of the linear relationship between head orientation and talker direction can extracted. This linear relationship for users sitting in different locations (users 2, 4, and 6 in FIG. 3A) can be compared across talker configurations to evaluate its consistency. A one-way ANOVA on the slopes showed no significant main effect of user's location (F_{2.24}=1.85, p=0.32). The slopes for all user locations are significantly smaller than 1 (p<0.0001 for all), suggesting significant undershooting of head orientation. There is no significant difference between the slopes and the measured slope 0.6 for the linear relationship model (p>0.16 for all). A one-way ANOVA on the intercepts show significant main effect of user locations (F_{2.24}=7.39, p=0.003). The intercept of user 4 is significantly different from 0 (t₂₄=4.31, p=0.0002), while no significant difference from 0 can be found for user 2 or 6 (p>0.4 for both). Pairwise comparison shows that the intercept of user 4 is significantly lower than that of user 2 (t₂₄=3.65, p=0.004) and that of user 6 (t₂₄=2.87, p=0.022). Thus, the relationship is approximately linear, but the intercept varies across different target talker layouts. The predicted target location based on fitted HMI is also shown to be closer to the truth location than the linear relationship model.

To evaluate if the fitted HMMs provide benefit in guiding the beamformer over the linear relationship model, the error of HMM and raw head orientation in predicting target location can be analyzed. When there are two possible target talkers (4-people sessions), the error of HMI prediction is significantly lower than that of the raw head orientation (t₇=3.28, p=0.013). To test if this benefit could be generalized to situation with more target talkers, the error can also be evaluated on 5-people sessions, which also show that HMM prediction has a lower error than the linear relationship model.

Since predicting real-time target location purely based on head orientation is challenging, a two-step sound source selection method is presented herein. The sound source selection method includes source registration and source selection. The sound source selection method may be implemented by an audio system, e.g., part of the audio controller 230 of the audio system 200. First, the locations of one or more sound sources may be registered through information from environment sensors 220, e.g., camera and microphone array. The locations may be registered relative to a user's location. The data store 235 of the audio system 200 may store values for the one or more sound sources and a location of each sound source. One or more sensors of the audio system 200 may measure the user's behavior, for example, one or more egocentric cameras, such as IMUs, can be used to detect the user's head movements. Based on the measured user's behavior, the audio controller 230 of the audio system 200 may determine a target sound source from the one or more sound sources using a hidden Markov model (HMM). In some embodiments, the HMI may determine one or more hidden states corresponding to the one or more sound sources, and calculate the relationship between the user's head orientation/movement and each of the hidden states. Based on the calculation, the HMM may predict a direction of the user's auditory attention, thereby determining the target sound source. A beamforming module 270 of the audio system 200 may then select auditory signals from the target sound source as an input to the user. The beamforming module 270 may be configured to enhance the sound from the target sound source and attenuate the signals from other sound sources. The source selection method based on HMM convert real-time head movements to a prediction of the target of a listener's auditory attention, and the performance of this method is significantly better than one purely based on head movement. The HMM filled the gap between the environment and user's intent and could reduce the impact of individual differences through parameters including state transfer matrix and emission functions.

In addition to group conversation, the source selection method based on HMI can also be generalized to other situations. The model only requires the locations of targets to be relatively fixed, so any type of fixed discrete sound source can be selected. As the undershoot problem of head orientation exists for various kinds of targets, HMI should provide similar benefit in source selection. Although the HMM assumption may not hold for human motion due to the continuity of body movement, involving head movement in other axis and adding an autoregressive component to represent this continuity may further improve the accuracy of HMM. Furthermore, the output from HMM could also be combined with other information to provide a final prediction of user's auditory attention. For example, eye tracking data could be combined with head tracking data to provide extra information. The head movement and eye gaze have been shown to be only weakly correlate in group conversation, thus selecting a target sound source may further comprise using the HMI based on the detected head movement and the eye gaze data. The input from multiple sensors could also be fused to cross-validate each other, so the HMM could be better tuned. For example, estimating the number of potential auditory targets could be greatly improved by including face tracking data, the number of clusters of head orientation, and the number of clusters of eye fixation.

Embodiments of the present disclosure are further related to an audio system for detecting a target sound source. The audio system may include a sensor array comprising one or more sensors (e.g., cameras, microphones, position sensors, etc.). The audio system may be integrated into a headset 100 and/or an audio system 200 that also includes at least one sensor of the one or more sensors. The sensor array captures information describing a local area of the headset 100 (or the audio system 200). The captured information may be, e.g., sounds within the local area, images of the local area (e.g., images of people in the local area, eye tracking information, images of portions of the user), position of the user within the local area, some other information describing the local area of the headset 100 (or the audio system 200), or some combination thereof. The capture information may include user's behavior, for example, head movements, eye gaze, etc. The sensor array may include, e.g., a plurality of acoustic sensors 180, the one or more imaging devices 130, the DCA, the PCA, the position sensor 190, or some combination thereof. The sensor array may be the sensor array 220 in the audio system 200. The audio system may further comprise a beamforming module, such as the beamforming module 270, which is configured to selectively analyze discrete sound sources and enhance a signal from a sound source.

The audio system is configured to register locations of one or more sound sources relative to a user's location; detect a head movement of the user; determine a target sound source from the one or more sound sources using a hidden Markov model (HMM) based on the detected head movement and the locations of the one or more sound sources; and then select auditory signals from the target sound source as an input to the user.

### Predicting Risk of Dementia by Tracking User Social Activity

A health monitoring system is presented herein for predicting risk of hearing loss and/or dementia by tracking a user's social activity. The health monitoring system monitors social interactions of the user to predict a risk of dementia/hearing loss for the use. The health monitoring system may be integrated into a headset or an audio system. However, in other embodiments, the health monitoring system may include different and/or additional components. Similarly, in some cases, functionality described with reference to the components of the health monitoring system can be distributed among the components in a different manner than is described here. For example, some or all of the functions of the health monitoring system may be performed by a remote server.

In some embodiments, the health monitoring system may include a sensor array comprising one or more sensors (e.g., cameras, microphones, position sensors, etc.). The health monitoring system may be integrated into a headset 100 and/or an audio system 200 that also includes at least one sensor of the one or more sensors. The sensor array captures information describing a local area of the headset 100 (or the audio system 200). The captured information may be, e.g., sounds within the local area, images of the local area (e.g., images of people in the local area, eye tracking information, images of portions of the user), position of the user within the local area, some other information describing the local area of the headset 100 (or the audio system 200), or some combination thereof. The sensor array may include, e.g., a plurality of acoustic sensors 180, the one or more imaging devices 130, the DCA, the PCA, the position sensor 190, or some combination thereof. The sensor array may be the sensor array 220 in the audio system 200.

The health monitoring system may process information from sensors on the headset 100, the audio system 200, and/or other sensors external to the headset (e.g., a position sensor on a watch worn by the user). The health monitoring system uses information collected by the headset to monitor social interaction of a user. The health monitoring system predicts a risk of dementia and/or hearing loss of the user using the amount of social interaction and a model (e.g., machine learned). The health monitoring system then can generate a recommendation for future social interaction of the user based in part on the predicted risk, and instructs the headset (or audio system) to present (e.g., via speakers and/or display) the recommendation to the user.

The health monitoring system may be configured to determine an amount of social interactions of the user with the other people for a given period of time based in part on the captured information. A social interaction of the user may include a conversation the user has with one or more other people or devices. The conversation may be in person or via a device (e.g., smartphone, headset 100, audio system 200, configured to handle calls, etc.). The health monitoring system may monitor sound sources in the local area and a voice of the user, and identify when the user enters a conversation with one or more users. The health monitoring system may count the number of social interactions the user has over a given time period (e.g., daily, or some other time metric). The health monitoring system may also track a length of one or more of the monitored conversations. The health monitoring system may also track a depth of one or more of the monitored conversations. Depth may be determined in part on the length and/or content of the conversation. For example, a conversation that is just an exchange of greetings is short and lacking depth relative to a conversation that is 15 minutes long. In some embodiments, the health monitoring system may track who the user speaks to in each conversation. In this manner, the health monitoring system may track diversity of people the user is interacting with.

In some embodiments, the health monitoring system may use the monitored interactions to estimate a level of social interaction of the user. The interaction may be a conversation, a physical gesture (e.g., tilting an ear towards a sound source, cupping an ear with a hand of the user, lack of user response to someone speaking the user's name, etc.). For example, the audio controller 150 may detect user interactions with other people. The audio controller 150 may use the tracked interactions to determine, e.g., how long each conversation is, a number of conversations, environment of the conversation and/or level of ambient noise during the conversation (e.g., in a loud restaurant or a quiet setting), a level of depth of a conversation (e.g., simply a greeting or something more substantial), categories of people spoken to (e.g., friends, family, spouse, stranger), identities of people spoken to, some other aspect relevant to tracking social interactions, or some combination thereof.

Moreover, in some embodiments, the health monitoring system may estimate if the user has hearing loss based on the social interactions. For example, of the interactions are consistently short, the user cuts short and/or minimizes conversations in environments with a lot of noise, user tilts head toward speaker and/or cups ear toward speaker (determined from position sensor data and/or images from cameras on the headset 100), may indicate the user has some level of hearing loss. In some embodiments, the health monitoring system may also monitor how user interactions differ based on access to visual cues. For example, how a user responds to someone calling their names when that person is in a field of view of the user, and how the user responds to someone calling their names when that person is not in a field of view of the user. Similarly, the health monitoring system may monitor how interactions differ based on sound source location relative to the user. For example, how a user responds to someone calling their names from different positions relative to the user (e.g., front, left side, right side, behind, etc.).

The health monitoring system may predict a risk of dementia of the user using the amount of social interaction and a model. The model may be, e.g., a trained machine learned model that outputs a predicted risk of dementia given an amount of social interaction. In other embodiments, the model is rule-based and maps specific combinations of social interaction to various predicted risks of dementia. In some embodiments, the model may predict risk of dementia based on the captured information from the sensor array.

The health monitoring system may generate a recommendation for future social interaction of the user based in part on the predicted risk. For example, the health monitoring system may generate a recommendation for interacting with at least three people a day for at least a threshold period of time at three different times of the day to help mitigate the risk of dementia. Likewise, if the health monitoring system has estimated that the user is experiencing hearing loss, the recommendation may also include a recommendation to visit an audiologist to check the user's hearing. The health monitoring system instructs the headset 100 (or audio system 200) to present (e.g., via display element and/or audio system) the recommendation to the user.

FIG. 4 is a flowchart of a method for predicting risk of dementia by tracking user social interaction 400, in accordance with one or more embodiments. The process shown in FIG. 4 may be performed by components of a health monitoring system. The health monitoring system may be integrated into a headset 100 and/or an audio system (e.g., audio system 200). Other entities may perform some or all of the steps in FIG. 4 in other embodiments. Embodiments may include different and/or additional steps, or perform the steps in different orders.

A health monitoring system captures 410, by one or more sensors (e.g., via the imaging device 130, acoustic sensor 180, position sensor 190, sensor array 220, etc.), information describing a social interaction of a user over a given time period.

The health monitoring system determines 420 an amount of the social interaction of the user for the given period of time based in part on the captured information. The social interaction may be a conversation, or a physical gesture. The health monitoring system may determine the amount of the social interaction based on frequency, length of time, number of times, level of depth of the social interaction, etc.

The health monitoring system predicts 430 a risk of dementia of the user using the amount of social interaction and a model. The model may output a predicted risk of dementia, e.g., a probability of developing dementia, based on the amount of social interaction. The model may be a trained machine learning model. In some embodiments, the health monitoring system may also predict 430 a risk of hearing loss using the model based on the determined amount of social interaction.

Based in part on the predicted risk, the health monitoring system 440 generates a recommendation for future social interaction of the user. The recommendation for future social interaction may include frequency, format, content, length of time, interaction method, etc., that is associated with the social interactions.

The health monitoring system presents 450 the recommendation to the user. The recommendation may be presented in way of video, audio, image, text, message, etc.

Audio System with Current/Voltage Sensor for Detecting Audio Leakage

Audio leakage in headphones, earbuds and hearables is a challenging issue which impacts user audio experience. The audio leakage could be caused by either users' misplacement of headphones on heads and earbuds/hearables in ears, and/or the cushions in headphones and the ear/hearable tips degradation after a long period of usage. Conventional audio leakage detection systems usually require microphones to capture sound and analyze the acoustic audio leakage. This can increase complexity of microphone placement and/or routing. In addition, it potentially couples with mechanical vibration from the render system.

Described herein is an audio system that detects audio leakage using electrical drive signals (e.g., current and/or voltage). The audio system may be integrated into earphones (e.g., headphones, in-ear devices, ear-buds) that operate with a fixed acoustic impedance load (i.e., a fixed acoustic volume). The audio system includes a speaker, an UV sensor, and controller. The speaker provides audio content to a user's ear. If audio leakage occurs (e.g., due to improper fit), the acoustic volume is no longer fixed, and changes the acoustic impedance load, which affects the current and/or voltage at the speaker. The I/V sensor senses current and/or voltage at the speaker. The controller uses a model and the sensed current and/or voltage to determine if audio leakage is present. If audio leakage is present, the audio system may alert the user of the audio system. This may allow the user to, e.g., re-adjust placement of the earphones to mitigate the audio leakage.

FIG. 5 illustrates an example audio system 500 with an I/V sensor 520 to detect audio leakage, in accordance with one or more embodiments. The audio system 500 may be integrated into a headset 100 and/or an audio system 200. The audio system 500 may be, e.g., headphones/earphones, in-ear devices, earbuds, some other devices that include a fixed acoustic volume, or some combination thereof. The audio system 500 may include a speaker 510, an I/V sensor 520 coupled with the speaker 510, and a controller 530. While FIG. 5 shows an example audio system 500 including one speaker 510, one UV sensor 520 and one controller 530, in other embodiments any number of these components may be included in the audio system 500. For example, there may be multiple speakers 510 each having an associated I/V sensor 520, with each speaker 510 and I/V sensor 520 communicating with the controller 530. In alternative configurations, different and/or additional components may be included in the audio system 500. Additionally, functionality described in conjunction with one or more of the components shown in FIG. 5 may be distributed among the components in a different manner than described in conjunction with FIG. 5 in some embodiments. For example, some or all of the functionality of the controller 530 may be provided by the headset 100 or the audio system 200. In some embodiments, the audio system 500 does not have an internal microphone used for detecting audio leakage. In some other embodiments, the audio system may not include any internal microphone at all.

The speaker 510 is integrated into the audio system that has a fixed acoustic impedance load. The speaker 510 is configured to provide audio content to the user. The speaker 510 may be driven by electrical drive signals. The electrical drive signal may be voltage and/or current.

The I/V sensor 520 may be coupled with the speaker 510. In some embodiments, the UV sensor 520 may be part of an amplifier used to drive the speaker 510. The one or more I/V sensor 520 may be configured to monitor the electrical drive signals provided to the speaker 510.

The controller 530 is configured to control components of the audio system 500. The controller 530 is configured to use the electrical drive signals detected by the UV sensor 520 and a model to determine a level of audio leakage. In some embodiments, the model maps various values of current and/or voltage to corresponding levels of audio leakage. In some embodiments, the model estimates one or more parameters of the speaker 510 using historical data and/or the monitored electrical drive signals in order to determine a level of audio leakage. The one or more parameters of the speaker 510 may include, voice coil, resistance, voice coil inductance, force factor, moving mass, radiation mass, speaker suspension stiffness, air volume compliance, speaker resistance, viscous resistance from audio leakage, etc.

Responsive to the level of audio leakage being above a threshold value, the controller 530 is configured to instruct the audio system 500 to alert a user to the audio leakage. The threshold value may be set such that, the audio leakage caused by cushion degradation and/or earphone misplacement results in an alert. The alert may be, e.g., an audio message to the user, or some other alert mechanism that the audio system 500 and/or some other system (e.g., one in which the audio system resides) is configured to provide (e.g., haptic feedback, displayed message, etc.).

Embodiments presented herein provide a simpler and more cost effect design. The audio system 500 is able to, e.g., ensure proper fit of the earphones. In addition to mitigating an audio leakage, proper fit can be important for, e.g., system calibration, providing a good seal for acoustic noise cancelation, and enhancement of bass. Also the audio system 500 described herein may help alert the user to when cushions in the earphones have degraded to a point where it is impacting performance.

A method for detecting audio leakage is described herein. An audio system may detect, by an UV senor, electrical drive signals that are provided to a specker of the audio system. The speaker is integrated into the audio system that has a fixed acoustic volume. Based on the detected electrical drive single, a controller of the audio system may determine a level of audio using a model. And responsive to the level of audio leakage being above a threshold value, the audio system may alert a user to the audio leakage.

### Augmenting Audio Background Based on Artificial Visual Background

Conventional video communication systems provide features that allow users to display an image or video as a background during a video communication. These virtual background features create a visual impression as if a user is physically at the location that is depicted in the background image or video, thus providing users with more privacy and better user experience. However, these conventional background features are limited to visual backgrounds. In this disclosure, systems and methods of augmenting audio background based on artificial visual background are presented.

A user participating in a video conference and/or preparing to join a video conference may select a background for presentation during the video conference. The background may be a static image and/or a dynamic image. The background may have associated acoustic parameters whose values describe effects a physical representation of the background image has on audio. The acoustic parameters may include, e.g., a reverberation time from a sound source to the headset for each of a plurality of frequency bands, a reverberant level for each frequency band, a direct to reverberant ratio for each frequency band, a direction of a direct sound from the sound source to the headset for each frequency band, an amplitude of the direct sound for each frequency band, a propagation time for the direct sound from the sound source to the headset, relative linear and angular velocities between the sound source and headset, a time of early reflection of a sound from the sound source to the headset, an amplitude of early reflection for each frequency band, a direction of early reflection, room mode frequencies, room mode locations, or some combination thereof. In some embodiments, where the background does not have associated acoustic parameter values, the communication device may use a machine learning model to estimate acoustic parameter values for the selected background and/or request acoustic parameter values (including acoustic parameters) from a server (e.g., conferencing server).

Embodiments of the present disclosure may include or be implemented in conjunction with an audio system (e.g., the audio system 200) that provides audio content. The audio system may be part of a headset (e.g., the headset 100). In some embodiments, the headset may be an artificial reality headset (e.g., presents content in virtual reality, augmented reality, and/or mixed reality). The audio system may use the method provided in embodiments herein to render audio background content to users through the headset. Audio background content is audio content that appears to originate from a particular physical representation, e.g., library, concert, beach, etc.

The audio system may be incorporated as part of a communication system in which artificial visual backgrounds in video calls are used to augment spatial audios. The communication system includes one or more communication devices, and may additionally include a server. A communication device may be, e.g., a computer, a tablet, a phone, a headset, an audio system, etc. The communication device includes a camera assembly (e.g., the imaging device 130), a microphone array (e.g., the acoustic sensor 180), a speaker assembly (e.g., the speaker 160), and a display (the display element 120). In some embodiments the communication device may include a local controller; alternatively, the controller may be implemented on a server of the communication system. The communication device may be used by a user to video conference with one or more other communication devices. In some cases, functionality described with reference to the components of the communication system can be distributed among the components in a different manner than is described here. For example, some or all of the functions of the controller may be performed by a remote server.

The camera assembly is configured to capture a video stream of the user. The camera assembly may include one or more cameras. A camera may be, e.g., a color camera. The camera assembly captures video stream in accordance with instructions from the controller.

The microphone array detects sounds in accordance with instructions from the controller. The microphone array includes a plurality of acoustic sensors (e.g., microphones). Each acoustic sensor is configured to detect sound and convert the detected sound into an electronic format (analog or digital). The microphone array may detect sounds from the user and outputs a corresponding audio stream. In some embodiments, a local area may have specific properties that affect how the user's speech is received at the microphone array. For example, a level of reverberation picked up by the microphone array is based in part on the physical settings of the local area.

The speaker assembly presents audio content to the user. In some embodiments, the audio content may be an audio stream, and in some embodiments, the audio content may be an audio stream with a specific acoustic effect. The speaker assembly may include a plurality of speakers that are configured to present an audio stream (e.g., associated with a video conference) to the user. For example, the audio stream may be audio from another user on the video conference call.

The display is configured to present video content to a user of the communication device. The video content may include one or more video streams associated with different communication devices participating in the video conference. In some embodiments, the video content may be one or more images associated with the different communications. The one or more images may be static images and/or dynamic images. The images may be in the format of PNG, JPEG, GIF, TIFF, PSD, EPS, etc. The display may be, e.g., a liquid crystal display, an organic light-emitting diode, or some other display.

The controller may instruct the camera assembly to capture a video stream and the microphone array to capture an audio stream from the local area. The controller is configured to receive the video stream and the audio stream, and the selected background. The controller may retrieve values for one or more acoustic parameters associated with the selected background from local storage and/or a server.

The controller may be configured to update the audio stream based on the meta data (e.g., the acoustic parameter values that are associated with the background) to generate an updated audio stream. For example, the controller may update the audio stream based in part on an RT60 value associated with the physical representation of the background (rather than the actual RT60 value of the local area where the user is located). In another example, the controller may update the audio stream to have the acoustic effects so that the updated audio stream sounds like being generated in the physical representation related to the background. Alternatively, the controller may be configured to provide one or more audio update options for the user to choose. The user may select one of the audio update options to generate the updated audio stream.

The controller may provide the video stream and updated audio stream to a communication device (or conference server which then distributes to other communication devices in the video conference). For example, a communication device may receive and combine the video stream and updated audio stream to generate an updated video stream. And because the updated audio stream is updated in accordance with the acoustic parameter values of the physical representation related to the background, the updated audio stream sounds as if it originated from the user being located in the physical representation of the background.

In some embodiments, the controller may instruct the communication device to send the audio stream, video stream, background (and associated acoustic parameter values if present) to the conferencing server. And the conferencing server updates the audio stream using the acoustic parameter values prior and distributes it along with the video stream (with the background) to other conference participants. In some embodiments, where the background does not have associated acoustic parameter values, the conferencing server may use a machine learning model to estimate acoustic parameter values for the selected background.

Alternatively, the controller may instruct the communication device to send the audio stream, video stream, background (and the associated acoustic parameters if present) to another communication device. And the other communication device updates the audio stream using the acoustic parameter values prior and presents it along with the video stream (with the background). In some embodiments, where the background does not have associated acoustic parameter values, the other communication device may use a machine learning model to estimate acoustic parameter values for the selected background and/or request it from a server (e.g., the conference server).

FIG. 6 is a flowchart of a method of augmenting audio background based on artificial visual background 600, in accordance with one or more embodiments. The process shown in FIG. 6 may be performed by components of a communication system. The communication system may include one or more communication devices, e.g., audio system 200 and/or a headset 100, and/or a server. Other entities may perform some or all of the steps in FIG. 6 in other embodiments. Embodiments may include different and/or additional steps, or perform the steps in different orders.

The communication system receives 610 an audio stream sent from a sound source and a background image. The background image is associated with one or more acoustic parameters, and the acoustic parameters may describe an acoustic effect that a physical representation related to the background image has on audio. In one embodiment, the audio stream may be captured by an audio system of a communication device and sent from the communication device to a server of the communication system. In another embodiments, the communication system may be integrated with a headset or some other device that captures the audio stream. In some embodiments, the background image may be sent from a communication device to the communication system; and in other embodiments, the background image may be stored at a server and selected by a user. In some cases, steps described with reference to the communication system can be performed in a different manner than is described here. For example, some or all of the steps in FIG. 6 may be performed by a remote server; alternatively, some or all of the steps in FIG. 6 may be performed by local communication devices.

The communication system updates 620 the audio stream based on the acoustic parameters to generate an updated audio stream. In some embodiments, updating the audio stream based in part on the acoustic parameters comprises determining the values of the one or more acoustic parameters associated with the background image. The communication system may use a machine learning model to estimate acoustic parameter values based on the selected background and/or request acoustic parameter values (including acoustic parameters) from a server (e.g., conferencing server).

The communication system provides 630 the updated audio stream to a communication device to present the updated audio stream so that the audio from the sound source in the updated audio stream has the acoustic effect, and the acoustic effect sounds as if the sound source is located in the physical representation related to the background image.

In some embodiments, the background image may include a static image, a dynamic image, a background video, or a plurality of images changing during the audio stream. The communication system may determine and update the one or more acoustic parameters associated with the background image as the background image changes during the audio stream. As such, the acoustic effect of the updated audio stream updates in accordance with the background image. And the updated audio stream sounds as if the location of the sound source is also changing in accordance with the physical representation related to the background image.

The communication system may also receive a video stream in addition to the audio stream. The video stream may be incorporated with the audio stream. The video stream may include a background image or an artificial visual background. The communication system may determine the one or more acoustic parameters associated with the background image or the artificial visual background, and update the audio stream based on the acoustic parameters to generate an updated audio stream. The communication system then combines the video stream with the updated audio stream to generate an updated video stream. The communication system sends the updated video stream to a communication device to present the updated video stream so that the audio from the sound source in the updated video stream sounds as if the sound source is located in the physical representation related to the background image or the artificial visual background.

In one example, a user is in their home office on a video conference. The user initially has no background on, and audio from the user as presented to others on the call sounds as if the user is speaking from their home office. The user then updates the background to be a concert hall. The communication system receives the background image, i.e., the concert hall image, and determines the acoustic parameters associated with the background image. The acoustic parameters describe the acoustic effect that a concert hall may have on audio. Based on the acoustic parameters, the communication system then modifies the audio stream to have the acoustic effect of the concert hall. As such, others on the call see the user with the concert hall background, but the user now also sounds like he physically is in the concert hall (when in actuality the user is still in the home office).

In another example, the same background image may be associated with one or more sets of acoustic parameters values, and each set of acoustic parameter values may be associated with a different acoustic effect. For example, a concert hall background image may be associated with acoustic effect of symphony, jazz, or choir, etc. The communication system may provide one or more audio updating options that correspond to the one or more sets of acoustic parameters. The communication system may present the one or more audio updating options to the user. The user may select one of the audio updating option based on the corresponding acoustic effects. The communication system then updates the audio stream with the user selected audio updating option (i.e., the selected acoustic effect).

In still another example, the background image may be not directly related to a physical location. The background image may have any kind of content. For example, the background image may include a group of cats, a cartoon figure, a movie scene, etc. In such cases, the background image may not be associated with any acoustic parameter. The communication system may use a machine learning model to determine the physical representation related to the background image and estimate the acoustic parameters values for the background. Alternatively, the communication system may request the acoustic parameter values (including acoustic parameters) from a server (e.g., conferencing server).

FIG. 7 is a system 700 that includes a headset 705, in accordance with one or more embodiments. In some embodiments, the headset 705 may be the headset 100 of FIG. 1A or the headset 105 of FIG. 1B. The system 700 may operate in an artificial reality environment (e.g., a virtual reality environment, an augmented reality environment, a mixed reality environment, or some combination thereof). The system 700 shown by FIG. 7 includes the headset 705, an input/output (I/O) interface 710 that is coupled to a console 715, the network 720, and the mapping server 725. While FIG. 7 shows an example system 700 including one headset 705 and one I/O interface 710, in other embodiments any number of these components may be included in the system 700. For example, there may be multiple headsets each having an associated I/O interface 710, with each headset and I/O interface 710 communicating with the console 715. In alternative configurations, different and/or additional components may be included in the system 700. Additionally, functionality described in conjunction with one or more of the components shown in FIG. 7 may be distributed among the components in a different manner than described in conjunction with FIG. 7 in some embodiments. For example, some or all of the functionality of the console 715 may be provided by the headset 705.

The headset 705 includes the display assembly 730, an optics block 735, one or more position sensors 740, and the DCA 745. Some embodiments of headset 705 have different components than those described in conjunction with FIG. 7. Additionally, the functionality provided by various components described in conjunction with FIG. 7 may be differently distributed among the components of the headset 705 in other embodiments, or be captured in separate assemblies remote from the headset 705.

The display assembly 730 displays content to the user in accordance with data received from the console 715. The display assembly 730 displays the content using one or more display elements (e.g., the display elements 120). A display element may be, e.g., an electronic display. In various embodiments, the display assembly 730 comprises a single display element or multiple display elements (e.g., a display for each eye of a user). Examples of an electronic display include: a liquid crystal display (LCD), an organic light emitting diode (OLED) display, an active-matrix organic light-emitting diode display (AMOLED), a waveguide display, some other display, or some combination thereof. Note in some embodiments, the display element 120 may also include some or all of the functionality of the optics block 735.

The optics block 735 may magnify image light received from the electronic display, corrects optical errors associated with the image light, and presents the corrected image light to one or both eyeboxes of the headset 705. In various embodiments, the optics block 735 includes one or more optical elements. Example optical elements included in the optics block 735 include: an aperture, a Fresnel lens, a convex lens, a concave lens, a filter, a reflecting surface, or any other suitable optical element that affects image light. Moreover, the optics block 735 may include combinations of different optical elements. In some embodiments, one or more of the optical elements in the optics block 735 may have one or more coatings, such as partially reflective or anti-reflective coatings.

Magnification and focusing of the image light by the optics block 735 allows the electronic display to be physically smaller, weigh less, and consume less power than larger displays. Additionally, magnification may increase the field of view of the content presented by the electronic display. For example, the field of view of the displayed content is such that the displayed content is presented using almost all (e.g., approximately 110 degrees diagonal), and in some cases, all of the user's field of view. Additionally, in some embodiments, the amount of magnification may be adjusted by adding or removing optical elements.

In some embodiments, the optics block 735 may be designed to correct one or more types of optical error. Examples of optical error include barrel or pincushion distortion, longitudinal chromatic aberrations, or transverse chromatic aberrations. Other types of optical errors may further include spherical aberrations, chromatic aberrations, or errors due to the lens field curvature, astigmatisms, or any other type of optical error. In some embodiments, content provided to the electronic display for display is pre-distorted, and the optics block 735 corrects the distortion when it receives image light from the electronic display generated based on the content.

The position sensor 740 is an electronic device that generates data indicating a position of the headset 705. The position sensor 740 generates one or more measurement signals in response to motion of the headset 705. The position sensor 190 is an embodiment of the position sensor 740. Examples of a position sensor 740 include: one or more IMUs, one or more accelerometers, one or more gyroscopes, one or more magnetometers, another suitable type of sensor that detects motion, or some combination thereof. The position sensor 740 may include multiple accelerometers to measure translational motion (forward/back, up/down, left/right) and multiple gyroscopes to measure rotational motion (e.g., pitch, yaw, roll). In some embodiments, an IMU rapidly samples the measurement signals and calculates the estimated position of the headset 705 from the sampled data. For example, the IMU integrates the measurement signals received from the accelerometers over time to estimate a velocity vector and integrates the velocity vector over time to determine an estimated position of a reference point on the headset 705. The reference point is a point that may be used to describe the position of the headset 705. While the reference point may generally be defined as a point in space, however, in practice the reference point is defined as a point within the headset 705.

The DCA 745 generates depth information for a portion of the local area. The DCA includes one or more imaging devices and a DCA controller. The DCA 745 may also include an illuminator. Operation and structure of the DCA 745 is described above with regard to FIG. 1A.

The audio system 750 provides audio content to a user of the headset 705. The audio system 750 is substantially the same as the audio system 200 describe above. The audio system 750 may comprise one or acoustic sensors, one or more transducers, and an audio controller. The audio system 750 may provide spatialized audio content to the user. In some embodiments, the audio system 750 may request acoustic parameters from the mapping server 725 over the network 720. The acoustic parameters describe one or more acoustic properties (e.g., room impulse response, a reverberation time, a reverberation level, etc.) of the local area. The audio system 750 may provide information describing at least a portion of the local area from e.g., the DCA 745 and/or location information for the headset 705 from the position sensor 740. The audio system 750 may generate one or more sound filters using one or more of the acoustic parameters received from the mapping server 725, and use the sound filters to provide audio content to the user.

The I/O interface 710 is a device that allows a user to send action requests and receive responses from the console 715. An action request is a request to perform a particular action. For example, an action request may be an instruction to start or end capture of image or video data, or an instruction to perform a particular action within an application. The I/O interface 710 may include one or more input devices. Example input devices include: a keyboard, a mouse, a game controller, or any other suitable device for receiving action requests and communicating the action requests to the console 715. An action request received by the I/O interface 710 is communicated to the console 715, which performs an action corresponding to the action request. In some embodiments, the I/O interface 710 includes an IMU that captures calibration data indicating an estimated position of the I/O interface 710 relative to an initial position of the I/O interface 710. In some embodiments, the I/O interface 710 may provide haptic feedback to the user in accordance with instructions received from the console 715. For example, haptic feedback is provided when an action request is received, or the console 715 communicates instructions to the I/O interface 710 causing the I/O interface 710 to generate haptic feedback when the console 715 performs an action.

The console 715 provides content to the headset 705 for processing in accordance with information received from one or more of: the DCA 745, the headset 705, and the I/O interface 710. In the example shown in FIG. 7, the console 715 includes an application store 755, a tracking module 760, and an engine 765. Some embodiments of the console 715 have different modules or components than those described in conjunction with FIG. 7. Similarly, the functions further described below may be distributed among components of the console 715 in a different manner than described in conjunction with FIG. 7. In some embodiments, the functionality discussed herein with respect to the console 715 may be implemented in the headset 705, or a remote system.

The application store 755 stores one or more applications for execution by the console 715. An application is a group of instructions, that when executed by a processor, generates content for presentation to the user. Content generated by an application may be in response to inputs received from the user via movement of the headset 705 or the I/O interface 710. Examples of applications include: gaming applications, conferencing applications, video playback applications, or other suitable applications.

The tracking module 760 tracks movements of the headset 705 or of the I/O interface 710 using information from the DCA 745, the one or more position sensors 740, or some combination thereof. For example, the tracking module 760 determines a position of a reference point of the headset 705 in a mapping of a local area based on information from the headset 705. The tracking module 760 may also determine positions of an object or virtual object. Additionally, in some embodiments, the tracking module 760 may use portions of data indicating a position of the headset 705 from the position sensor 740 as well as representations of the local area from the DCA 745 to predict a future location of the headset 705. The tracking module 760 provides the estimated or predicted future position of the headset 705 or the I/O interface 710 to the engine 765.

The engine 765 executes applications and receives position information, acceleration information, velocity information, predicted future positions, or some combination thereof, of the headset 705 from the tracking module 760. Based on the received information, the engine 765 determines content to provide to the headset 705 for presentation to the user. For example, if the received information indicates that the user has looked to the left, the engine 765 generates content for the headset 705 that mirrors the user's movement in a virtual local area or in a local area augmenting the local area with additional content. Additionally, the engine 765 performs an action within an application executing on the console 715 in response to an action request received from the I/O interface 710 and provides feedback to the user that the action was performed. The provided feedback may be visual or audible feedback via the headset 705 or haptic feedback via the I/O interface 710.

The network 720 couples the headset 705 and/or the console 715 to the mapping server 725. The network 720 may include any combination of local area and/or wide area networks using both wireless and/or wired communication systems. For example, the network 720 may include the Internet, as well as mobile telephone networks. In one embodiment, the network 720 uses standard communications technologies and/or protocols. Hence, the network 720 may include links using technologies such as Ethernet, 802.11, worldwide interoperability for microwave access (WiMAX), 2G/3G/4G mobile communications protocols, digital subscriber line (DSL), asynchronous transfer mode (ATM), InfiniBand, PCI Express Advanced Switching, etc. Similarly, the networking protocols used on the network 720 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), etc. The data exchanged over the network 720 can be represented using technologies and/or formats including image data in binary form (e.g., Portable Network Graphics (PNG)), hypertext markup language (HTML), extensible markup language (XML), etc. In addition, all or some of links can be encrypted using conventional encryption technologies such as secure sockets layer (SSL), transport layer security (TLS), virtual private networks (VPNs), Internet Protocol security (IPsec), etc.

The mapping server 725 may include a database that stores a virtual model describing a plurality of spaces, wherein one location in the virtual model corresponds to a current configuration of a local area of the headset 705. The mapping server 725 receives, from the headset 705 via the network 720, information describing at least a portion of the local area and/or location information for the local area. The user may adjust privacy settings to allow or prevent the headset 705 from transmitting information to the mapping server 725. The mapping server 725 determines, based on the received information and/or location information, a location in the virtual model that is associated with the local area of the headset 705. The mapping server 725 determines (e.g., retrieves) one or more acoustic parameters associated with the local area, based in part on the determined location in the virtual model and any acoustic parameters associated with the determined location. The mapping server 725 may transmit the location of the local area and any values of acoustic parameters associated with the local area to the headset 705.

One or more components of system 700 may contain a privacy module that stores one or more privacy settings for user data elements. The user data elements describe the user or the headset 705. For example, the user data elements may describe a physical characteristic of the user, an action performed by the user, a location of the user of the headset 705, a location of the headset 705, an HRTF for the user, etc. Privacy settings (or “access settings”) for a user data element may be stored in any suitable manner, such as, for example, in association with the user data element, in an index on an authorization server, in another suitable manner, or any suitable combination thereof.

A privacy setting for a user data element specifies how the user data element (or particular information associated with the user data element) can be accessed, stored, or otherwise used (e.g., viewed, shared, modified, copied, executed, surfaced, or identified). In some embodiments, the privacy settings for a user data element may specify a “blocked list” of entities that may not access certain information associated with the user data element. The privacy settings associated with the user data element may specify any suitable granularity of permitted access or denial of access. For example, some entities may have permission to see that a specific user data element exists, some entities may have permission to view the content of the specific user data element, and some entities may have permission to modify the specific user data element. The privacy settings may allow the user to allow other entities to access or store user data elements for a finite period of time.

The privacy settings may allow a user to specify one or more geographic locations from which user data elements can be accessed. Access or denial of access to the user data elements may depend on the geographic location of an entity who is attempting to access the user data elements. For example, the user may allow access to a user data element and specify that the user data element is accessible to an entity only while the user is in a particular location. If the user leaves the particular location, the user data element may no longer be accessible to the entity. As another example, the user may specify that a user data element is accessible only to entities within a threshold distance from the user, such as another user of a headset within the same local area as the user. If the user subsequently changes location, the entity with access to the user data element may lose access, while a new group of entities may gain access as they come within the threshold distance of the user.

The system 700 may include one or more authorization/privacy servers for enforcing privacy settings. A request from an entity for a particular user data element may identify the entity associated with the request and the user data element may be sent only to the entity if the authorization server determines that the entity is authorized to access the user data element based on the privacy settings associated with the user data element. If the requesting entity is not authorized to access the user data element, the authorization server may prevent the requested user data element from being retrieved or may prevent the requested user data element from being sent to the entity. Although this disclosure describes enforcing privacy settings in a particular manner, this disclosure contemplates enforcing privacy settings in any suitable manner.

In some embodiments, the system 700 register sound sources and/or detect user behaviors by using the headset 705, and/or other components. Based on the detected information, the system 700 may determine a target sound source and select auditory signals from the target sound source as an input to the user. The system 700 may capture information describing a social interaction of a user, determine an amount of the user's social interaction, and predict a risk of dementia and/hearing loss of the user. Additionally, the system 700 may be configured to detect an audio leakage of the headset 705. Further, the system 700 may augment audio background based on an artificial visual background in a video stream.

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
registering locations of one or more sound sources relative to a user's location;
detecting, by one or more sensors, a head movement of the user;
determining a target sound source from the one or more sound sources using a hidden Markov model (HMM) based on the detected head movement and the locations of the one or more sound sources; and
selecting auditory signals from the target sound source as an input to the user.

2. The method of claim 1, wherein determining the target sound source comprising:
calculating a relationship between the head movement and each of the one or more sound sources based on the HMM;
determining a direction of the user's auditory attention based on the calculated relationship; and
determining the target sound source based on the direction of the user's auditory attention and the locations of the one or more sound sources.

3. The method of claim 1, further comprising:
determining, by the one or more sensors, eye tracking information of the user; and
determining the target sound source based on the detected head movement and the determined eye tracking information.

4. The method of claim 1, wherein selecting auditory signals from the target sound source as an input to the user comprises: enhancing the auditory signals from the target sound source and attenuating other auditory signals from other sound sources.

5. The method of claim 1, wherein the one or more sensors comprise one or more of camera, microphone, and position sensor.

6. The method of claim 1, further comprising:
capturing, by the one or more sensors, information describing a social interaction of a user over a given period of time;
determining an amount of the social interaction of the user for the given period of time based in part on the captured information;
predicting a risk of dementia of the user using the amount of social interaction and a model;
generating a recommendation for future social interaction of the user based in part on the predicted risk; and
presenting the recommendation to the user.

7. The method of claim 6, wherein the model is a machine learning model.

8. The method of claim 6, wherein the model is a rule-based model that maps an amount of social interaction to a predetermined risk of dementia.

9. The method of claim 6, further comprising: predicting a risk of hearing loss of the user based on the amount of social interaction.

10. The method of claim 6, wherein the amount of social interaction includes one or more of length of time, number of times, level of depth of the social interaction.

11. The method of claim 6, wherein the recommendation for future social interaction includes one or more of frequency, format, content, length of time, interaction method of the future social interaction.

12. A method comprising:
detecting, via an UV sensor of an audio system, an electrical drive signal provided to a speaker of the audio system having a fixed acoustic volume;
determining, via a controller of the audio system, a level of audio leakage based on the detected electrical drive signal and a model; and
responsive to the level of audio leakage being above a threshold value, alerting, via the audio system, a user to the audio leakage.

13. The method of claim 12, wherein determining the level of audio leakage comprising using the model to map the detected electrical drive signal to a corresponding level of audio leakage.

14. The method of claim 12, wherein determining the level of audio leakage comprising using the model to estimate one or more parameters of the speaker based on historical data; and
determining the level of audio leakage based on the one or more parameters and the detected electrical drive signal.

15. The method of claim 14, wherein the one or more parameters include: voice coil, resistance, voice coil inductance, force factor, moving mass, radiation mass, speaker suspension stiffness, air volume compliance, speaker resistance, and viscous resistance from audio leakage.

16. A method comprising:
receiving an audio stream from a sound source and a background image that is associated with one or more acoustic parameters, the acoustic parameters describing an acoustic effect a physical representation related to the background image has on audio;
updating the audio stream based on the one or more acoustic parameters to generate an updated audio stream; and
providing the updated audio stream to a communication device, wherein the communication device presents the updated audio stream having the acoustic effect as if the sound source is located in the physical representation related to the background image.

17. The method of claim 16, further comprising:
determining values of the one or more acoustic parameters; and
determining the acoustic effect based on the values of the one or more acoustic parameters.

18. The method of claim 17, wherein determining values of the one or more acoustic parameters comprising: using a machine learning model to estimate the values based on the background image.

19. The method of claim 17, wherein determining values of the one or more acoustic parameters comprising: requesting the values from a server.

20. The method of claim 16, wherein the background image is an artificial visual background.
